(19) United States
(12) Patent Application Publication
(10) Pub. No.: US 2015/0366608 A1
(43) Pub. Date: Dec. 24, 2015

(54) MEDICAL DEVICE FOR SYMPATHETIC NERVE ABLATION WITH PRINTED COMPONENTS

(71) Applicant: BOSTON SCIENTIFIC SCIMED, INC., MAPLE GROVE, MN (US)

(72) Inventors: JAN WEBER, MAASTRICHT (NL); JEFFREY S. LINDQUIST, MAPLE GROVE, MN (US); STEVEN R. LARSEN, LINO LAKES, MN (US); ERIC M. PETERSEN, MAPLE GROVE, MN (US); MARTIN R. WILLARD, BURNSVILLE, MN (US)

(73) Assignee: BOSTON SCIENTIFIC SCIMED, INC., MAPLE GROVE, MN (US)

(21) Appl. No.: 14/745,311

(22) Filed: Jun. 19, 2015

Related U.S. Application Data
(60) Provisional application No. 62/015,140, filed on Jun. 20, 2014.

(51) Int. Cl. A61B 18/14; A61B 18/04; A61B 18/16

(52) U.S. Cl. CPC A61B 18/1492; A61B 18/16; A61B 18/04; A61B 2018/00577

(57) ABSTRACT

A medical device for sympathetic nerve ablation may include an elongate shaft and an expandable member. A printed ablation electrode assembly may be disposed on an outer surface of the expandable member, the printed ablation electrode assembly including a positive electrical pathway and a ground electrical pathway printed directly on the outer surface of the expandable member. A temperature sensor may be printed directly on the outer surface of the expandable member. A method of manufacturing a medical device for sympathetic nerve ablation may include printing a conductive ink network directly on a surface of a polymeric balloon material in a flat configuration, printing at least one temperature sensor directly on the surface of the polymeric balloon material, forming the polymeric balloon material into an inflatable balloon, and attaching the inflatable balloon to an elongate catheter shaft.

### CROSS-REFERENCE TO RELATED APPLICATIONS

This application claims priority under 35 U.S.C. §119 to U.S. Provisional Application Ser. No. 62/015,140, filed Jun. 20, 2014, the entirety of which is incorporated herein by reference.

### TECHNICAL FIELD

The present disclosure pertains to medical devices, and methods for manufacturing medical devices. More particularly, the present disclosure pertains to medical devices for sympathetic nerve ablation.

### BACKGROUND

A wide variety of intracorporeal medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

In a first aspect, a medical device for sympathetic nerve ablation at one or more treatment sites may comprise an elongate catheter shaft having a guidewire lumen extending therethrough and an expandable member disposed adjacent a distal end, a printed ablation electrode assembly disposed on an outer surface of the expandable member, the printed ablation electrode assembly including a positive electrical pathway printed directly on the outer surface of the expandable member and a ground electrical pathway printed directly on the outer surface of the expandable member, and a temperature sensor printed directly on the outer surface of the expandable member.

In addition or alternatively, and in a second aspect, the printed ablation electrode assembly includes at least one positive electrode printed directly on the outer surface of the expandable member in electrical communication with the positive electrical pathway, and at least one ground electrode printed directly on the outer surface of the expandable member in electrical communication with the ground electrical pathway.

In addition or alternatively, and in a third aspect, the temperature sensor is printed directly on the outer surface of the expandable member between the at least one positive electrode and the at least one ground electrode.

In addition or alternatively, and in a fourth aspect, the medical device includes a positive electrical pathway printed directly on an outer surface of the elongate catheter shaft and in electrical communication with the positive electrical pathway printed directly on the outer surface of the expandable member, and a ground electrical pathway printed directly on the outer surface of the elongate catheter shaft and in electrical communication with the ground electrical pathway printed directly on the outer surface of the expandable member.

In addition or alternatively, and in a fifth aspect, the medical device includes a second temperature sensor printed directly on the outer surface of the expandable member and spaced distally from the printed ablation electrode assembly.

In addition or alternatively, and in a sixth aspect, the medical device includes a second positive electrode distinct from the at least one positive electrode, and a second ground electrode distinct from the at least one ground electrode. The second positive electrode and the second ground electrode distinct are disposed proximally of the at least one positive electrode and the at least one ground electrode, and are configured to operate at a reduced power compared to the at least one positive electrode and the at least one ground electrode.

In addition or alternatively, and in a seventh aspect, the expandable member is an inflatable balloon.

In addition or alternatively, and in an eighth aspect, the medical device may include at least one lumen formed within a wall of the inflatable balloon under and longitudinally aligned with each of the at least one positive electrode and the at least one ground electrode.

In addition or alternatively, and in a ninth aspect, each of the at least one lumen formed within the wall of the inflatable balloon includes at least one discharge aperture through the outer surface of the inflatable balloon.

In addition or alternatively, and in a tenth aspect, the temperature sensor is a self-regulating positive temperature coefficient (PTC) electrode printed directed on the outer surface of the expandable member connecting the positive electrical pathway and the ground electrical pathway.

In addition or alternatively, and in an eleventh aspect, a medical device for sympathetic nerve ablation at one or more treatment sites may comprise an elongate catheter shaft having a guidewire lumen extending therethrough and an inflatable balloon disposed adjacent a distal end, a positive electrical pathway printed directly on an outer surface of the inflatable balloon in a first recessed channel formed in the outer surface of the inflatable balloon, a ground electrical pathway printed directly on the outer surface of the inflatable balloon in a second recessed channel formed in the outer surface of the inflatable balloon, and a temperature sensor printed directly on the outer surface of the inflatable balloon.

In addition or alternatively, and in a twelfth aspect, the temperature sensor is printed directly on the outer surface of the inflatable balloon in a third recessed channel formed in the outer surface of the inflatable balloon.

In addition or alternatively, and in a thirteenth aspect, the temperature sensor is a self-regulating positive temperature coefficient (PTC) electrode printed directed on the outer surface of the inflatable balloon connecting the positive electrical pathway and the ground electrical pathway.

In addition or alternatively, and in a fourteenth aspect, the medical device includes at least one positive electrode printed directly on the outer surface of the inflatable balloon in electrical communication with the positive electrical pathway, and at least one ground electrode printed directly on the outer surface of the inflatable balloon in electrical communication with the ground electrical pathway.

In addition or alternatively, and in a fifteenth aspect, the temperature sensor is printed directly on the outer surface of the inflatable balloon between the at least one positive electrode and the at least one ground electrode.

In addition or alternatively, and in a sixteenth aspect, a method of manufacturing a medical device for sympathetic nerve ablation is disclosed. The method includes positioning a polymeric balloon material in a flat configuration, printing a conductive ink network directly on a surface of the polymeric balloon material in the flat configuration, and printing at least one temperature sensor directly on the surface of the polymeric balloon material. The method also includes forming the polymeric balloon material into an inflatable balloon, and attaching the inflatable balloon to an elongate catheter shaft.

In addition or alternatively, and in a seventeenth aspect, the method includes, before printing the conductive ink network, forming at least one recessed channel in the surface of the polymeric balloon material. The conductive ink network is printed within the at least one recessed channel.

In addition or alternatively, and in an eighteenth aspect, the method includes, before printing the conductive ink network, printing a polymer guide on the surface of the polymeric balloon material. The conductive ink network is printed within the polymer guide.

In addition or alternatively, and in a nineteenth aspect, the method includes, before forming the polymeric balloon material into the inflatable balloon, curing the conductive ink network.

In addition or alternatively, and in a twentieth aspect, the conductive ink network is formed from a nanoparticle suspension having metallic nanoparticles encapsulated by an organic binder.

The above summary of some embodiments, aspects, and/or examples is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a schematic view of an example medical system;

FIG. 2 is a schematic view of an example expandable member;

FIG. 3 is a schematic view of an example expandable member;

FIG. 4 is a partial cross-sectional view of FIG. 3;

FIG. 5 is a schematic view of an example expandable member;

FIG. 6 is a partial cross-sectional view of FIG. 5;

FIG. 7 is a schematic view of an example expandable member;

FIG. 8 is a partial cross-sectional view of FIG. 7;

FIG. 9 is a schematic view of an example expandable member;

FIG. 10 is a schematic view of an example expandable member;

FIG. 11 is a schematic view of an example expandable member;

FIG. 12 is a partial cross-sectional view of an example expandable member;

FIG. 13 is a partial cross-sectional view of an example expandable member; and

FIG. 14 illustrates an example method of manufacturing a medical device.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the claimed invention. Those skilled in the art will recognize that the various elements described and/or shown may be arranged in various combinations and configurations without departing from the scope of the disclosure. The detailed description and drawings illustrate example embodiments of the claimed invention.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (i.e., having the same function or result). In many instances, the term “about” may include numbers that are rounded to the nearest significant figure. Other uses of the term “about” (i.e., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to effect such feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangable with each other to form other additional embodiments or to complement and/or enrich the described embodiment(s), as would be understood by one of ordinary skill in the art.

Certain treatments are aimed at the temporary or permanent interruption or modification of select nerve function. In some embodiments, the nerves may be sympathetic nerves. One example treatment is renal nerve ablation, which is sometimes used to treat conditions such as or related to hypertension, congestive heart failure, diabetes, or other conditions impacted by high blood pressure or salt retention. The kidneys produce a sympathetic response, which may increase the undesired retention of water and/or sodium. The result of the sympathetic response, for example, may be an increase in blood pressure. Ablating some of the nerves running to the kidneys (e.g., disposed adjacent to or otherwise along the renal arteries) may reduce or eliminate this sympathetic response, which may provide a corresponding reduction in the associated undesired symptoms (e.g., a reduction in blood pressure).

Some embodiments of the present disclosure relate to a power generating and control apparatus, often for the treatment of targeted tissue in order to achieve a therapeutic effect. In some embodiments, the target tissue is tissue containing or proximate to nerves. In other embodiments, the target tissue is sympathetic nerves, including, for example, sympathetic nerves disposed adjacent to blood vessels. In still other embodiments the target tissue is luminal tissue, which may further comprise diseased tissue such as that found in arterial disease.

In some embodiments of the present disclosure, the ability to deliver energy in a targeted dosage may be used for nerve tissue in order to achieve beneficial biologic responses. For example, chronic pain, urologic dysfunction, hypertension, and a wide variety of other persistent conditions are known to be affected through the operation of nervous tissue. For example, it is known that chronic hypertension that may not be responsive to medication may be improved or eliminated by disabling excessive nerve activity proximate to the renal arteries. It is also known that nervous tissue does not naturally possess regenerative characteristics. Therefore it may be possible to beneficially affect excessive nerve activity by disrupting the conductive pathway of the nervous tissue. When disrupting nerve conductive pathways, it is particularly advantageous to avoid damage to neighboring nerves or organ tissue. The ability to direct and control energy dosage is well-suited to the treatment of nerve tissue. Whether in a heating or ablating energy dosage, the precise control of energy delivery as described and disclosed herein may be directed to the nerve tissue. Moreover, directed application of energy may suffice to target a nerve without the need to be in exact contact, as would be required when using a typical ablation probe. For example, eccentric heating may be applied at a temperature high enough to denature nerve tissue without causing ablation and without requiring the piercing of luminal tissue. However, it may also be desirable to configure the energy delivery surface of the present disclosure to pierce tissue and deliver ablating energy similar to an ablation probe with the exact energy dosage being controlled by a power control and generation apparatus.

In some embodiments, efficacy of the denervation treatment can be assessed by measurement before, during, and/or after the treatment to tailor one or more parameters of the treatment to the particular patient or to identify the need for additional treatments. For instance, a denervation system may include functionality for assessing whether a treatment has caused or is causing a reduction in neural activity in a target or proximate tissue, which may provide feedback for adjusting parameters of the treatment or indicate the necessity for additional treatments.

Many of the devices and methods described herein are discussed relative to renal nerve ablation and/or modulation. However, it is contemplated that the devices and methods may be used in other treatment locations and/or applications where sympathetic nerve modulation and/or other tissue modulation including heating, activation, blocking, disrupting, or ablation are desired, such as, but not limited to: blood vessels, urinary vessels, or in other tissues via trocar and cannula access. For example, the devices and methods described herein can be applied to hyperplastic tissue ablation, cardiac ablation, pain management, pulmonary vein isolation, pulmonary vein ablation, tumor ablation, benign prostatic hyperplasia therapy, nerve excitation or blocking or ablation, modulation of muscle activity, hyperthermia or other warming of tissues, etc. The disclosed methods and apparatus can be applied to any relevant medical procedure, involving both human and non-human subjects. The term modulation refers to ablation and other techniques that may alter the function of affected nerves and other tissue.

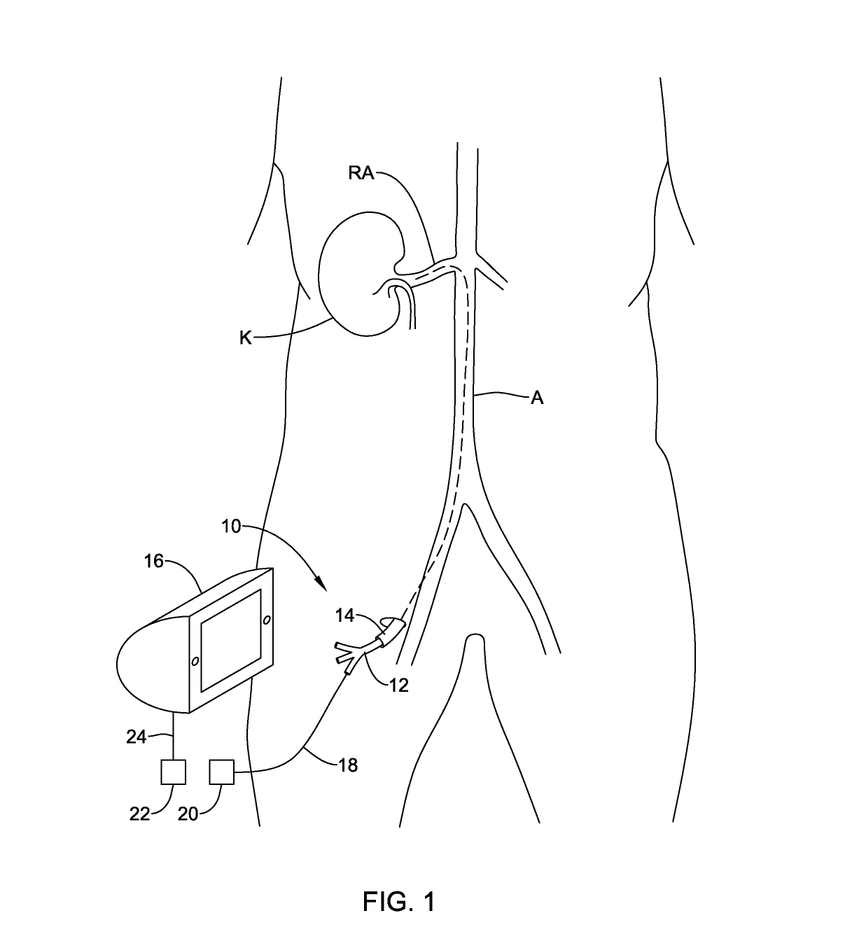

FIG. 1 is a schematic view of an example sympathetic nerve ablation system 10. System 10 may include a medical device 12 for sympathetic nerve ablation. In some embodiments, a medical device 12 may be used to ablate nerves (e.g., renal nerves) disposed adjacent to the kidney K (e.g., renal nerves disposed about a renal artery RA). In use, a medical device 12 may be advanced through a blood vessel such as the aorta A to a position within the renal artery RA. This may include advancing a medical device 12 through a guide sheath or catheter 14. When positioned as desired, a medical device 12 may be activated to activate one or more electrodes (not shown). This may include operatively and/or electrically coupling a medical device 12 to a control unit 16, which may include an RF generator, so as to supply the desired activation energy to the electrodes. For example, the medical device 12 may include a wire or conductive member 18 with a first connector 20 that can be connected to a second connector 22 on the control unit 16 and/or a wire 24 coupled to the control unit 16. In at least some embodiments, the control unit 16 may also be utilized to supply/receive the appropriate electrical energy and/or signal to activate one or more sensors disposed at or near a distal end of the medical device 12. When suitably activated, the one or more electrodes may be capable of ablating tissue (e.g., sympathetic nerves) as described below and the one or more sensors may be used to detect desired physical and/or biological parameters.

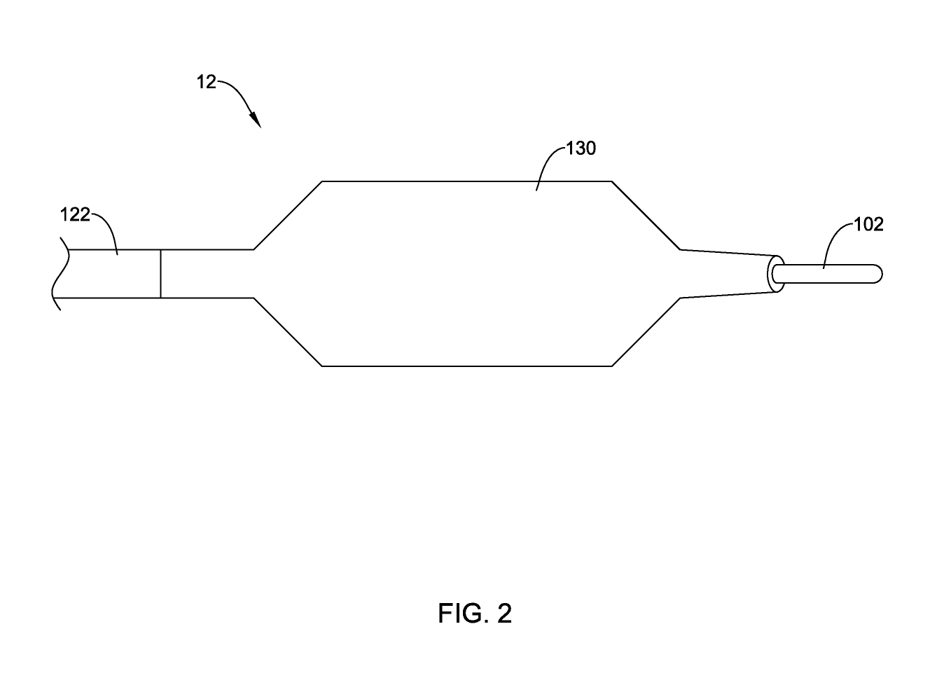

In some embodiments, a medical device 12 may include an elongate tubular member or catheter shaft 122 having a guidewire lumen extending therethrough, as shown in FIG. 2. In some embodiments, the elongate tubular member or catheter shaft 122 may be configured to be slidingly advanced over a guidewire 102 or other elongate medical device to a target site. In some embodiments, the elongate tubular member or catheter shaft 122 may be configured to be slidingly advanced within a guide sheath or catheter 14 to a target site. In some embodiments, the elongate tubular member or catheter shaft 122 may be configured to be advanced to a target site over a guidewire 102, within a guide sheath or catheter 14, or a combination thereof. An expandable member 130 may be disposed at, on, about, adjacent, or near a distal region or a distal end of the elongate tubular member or catheter shaft 122. In some embodiments, the expandable member 130 may be a compliant or a non-compliant polymeric inflatable balloon. Throughout the disclosure, the expandable member 130 is generally illustrated in the figures as an inflatable balloon for simplicity, but the skilled person will recognize that the expandable member 130 may take other forms such as, but not limited to, a scaffold, a cage, a stent, a plurality of struts, an expandable foam or sponge, or other suitable constructs. In some embodiments, the expandable member 130 may be capable of shifting between an unexpanded configuration and an expanded configuration. In some embodiments, the expandable member 130 may be expandable to a plurality of different sizes in the expanded configuration. In some embodiments, an expandable member 130 may be capable of shifting between a first geometric configuration and a second geometric configuration. In some embodiments, a medical device 12 and/or an expandable member 130 may include a tip section configured to bend laterally against (or to contact) a vessel wall at the target site upon activation or upon shifting to the second geometric configuration. In some embodiments, such a tip section may be activated or actuated by any suitable means, such as, but not limited to, electroactive polymers or shape memory materials such as nitinol (nickel-titanium alloy).

In some embodiments, a conductive ink network may be formed, deposited, or positioned on an outer surface of an expandable member 130 and/or an elongate tubular member or catheter shaft 122. In some embodiments, a conductive ink network may be printed directly on an outer surface of an expandable member 130 and/or an elongate tubular member or catheter shaft 122 using an ink jet printer, technology, and/or process. Other suitable processes, including, but not limited to ink-roller, pad printing, aerosol jet, or other ink-application processes are also considered and/or contemplated.

For the purpose of this disclosure, a conductive ink may be defined as a nanoparticle suspension, wherein nanoparticles are encapsulated by an organic binder or ligand to prevent clumping. In some embodiments, the nanoparticles may be metallic, such as, but not limited to gold, silver, platinum, and the like. In some embodiments, the organic binder or ligand may include, but is not limited to, nitrates, ammonium, acetates, sulfates, sulfides, and the like. The conductivity of these systems may be poor because ligands prevent electron transfer which is essential for electrical conductivity. In order to obtain electrical conductivity, the nanoparticle suspension is heated to decompose and/or “bake off” the organic binder or ligands. Some nanoparticles may have a high surface energy, such that the nanoparticles begin to coalesce as soon as the organic binder or ligands begin to evaporate. An interconnected matrix of nanoparticles begins to form as the organic binder or ligands is “baked off”, or decomposed with heat exposure. In some cases, a nanoparticle suspension may be heated to over 200 degrees C. to decompose a selected organic binder. In some embodiments, an organic binder may be selected which decomposes at relatively low temperatures of less than 100 degrees C. In some situations, a practitioner may mix a base ink with a co-solvent system (e.g., DMF, THF, acetone, xylene, or other solvent that will not dissolve the organic binder) to achieve suitable deposition and drying processes.

In some embodiments, a conductive ink network may be formed using a reactive ink system. Reactive inks do not involve discrete suspended nanoparticles, but instead include a metallic particle bound to something else, such as a ligand or a complexing agent. In one non-limiting example, silver atoms or particles may be bound to ammonia forming an ammoniacal compound or a metal ammine complex. When mixed with a reducing agent under specific conditions, an ammoniacal compound or a metal ammine complex may react with the reducing agent to produce, form, and/or deposit a metallic trace. In some situations, these systems may be advantageous because the electrical traces can be sintered at about 80 degrees C. to about 300 degrees C., which temperatures are generally compatible with inflatable balloon materials (for example, in a temperature range up to 180 degrees C.).

In some embodiments, a conductive ink network may be formed using masking and vacuum deposition of an electrically conductive material. The masking may then be removed to leave the conductive ink network behind on the surface of the medical device.

In some embodiments, a carbon filler may be added to the conductive ink network to further enhance conductivity. In some embodiments, one or more of several types of carbon-based materials, such as graphite, graphene, CNT, or nanobuds, which may be dry-printed on the polymer substrate of the expandable member 130 and/or the elongate tubular member or catheter shaft 122.

In some embodiments, a printed ink may be used as a “strike layer” which is then electroplated to form the conductive pathways of a conductive ink network. Such a process may increase conductivity without using high sintering temperatures.

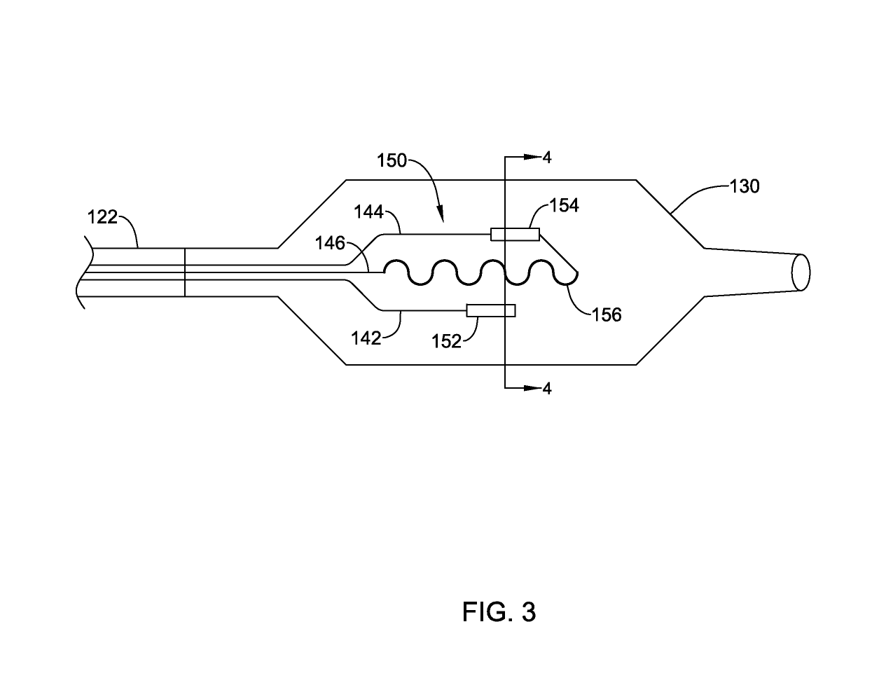

FIG. 3 illustrates an example expandable member 130 having a printed ablation electrode assembly 150 disposed on an outer surface of the expandable member 130. In some embodiments, a printed ablation electrode assembly 150 may include a positive electrical pathway 142 printed directly on the outer surface of the expandable member 130. In some embodiments, the positive electrical pathway 142 may be electrically coupled to a corresponding electrical pathway on the elongate tubular member or catheter shaft 122. In some embodiments, the positive electrical pathway 142 may be electrically coupled to a corresponding electrical pathway printed directly on the elongate tubular member or catheter shaft 122. In some embodiments, a printed ablation electrode assembly 150 may include a negative or ground electrical pathway 144 printed directly on the outer surface of the expandable member 130. In some embodiments, the negative or ground electrical pathway 144 may be electrically coupled to a corresponding electrical pathway on the elongate tubular member or catheter shaft 122. In some embodiments, the negative or ground electrical pathway 144 may be electrically coupled to a corresponding electrical pathway printed directly on the elongate tubular member or catheter shaft 122.

In some embodiments, one or more of the corresponding electrical pathways (i.e., a positive electrical pathway, a negative or ground electrical pathway, etc.) on the elongate tubular member or catheter shaft 122 may be printed directly on an outer surface of the elongate tubular member or catheter shaft 122. In other words, in some embodiments, the medical device 12 may include a positive electrical pathway printed directly on an outer surface of the elongate tubular member or catheter shaft 122 and in electrical communication with the positive electrical pathway printed directly on the outer surface of the expandable member 130. Similarly, in some embodiments, the medical device 12 may include a ground electrical pathway printed directly on the outer surface of the elongate tubular member or catheter shaft 122 and in electrical communication with the ground electrical pathway printed directly on the outer surface of the expandable member 130.

In some embodiments, a printed ablation electrode assembly 150 may include at least one positive electrode 152 printed directly on the outer surface of the expandable member 130 in electrical communication with the positive electrical pathway 142. In some embodiments, a printed ablation electrode assembly 150 may include at least one negative or ground electrode 154 printed directly on the outer surface of the expandable member 130 in electrical communication with the negative or ground electrical pathway 144. In some embodiments, a printed ablation electrode assembly 150 may include at least one pair of bipolar electrodes.

In some embodiments, a printed ablation electrode assembly 150 may have an asymmetric arrangement of the at least one positive electrode 152 and the at least one negative or ground electrode 154 about a central axis of the medical device 12. In some embodiments, a printed ablation electrode assembly 150 may have a symmetric arrangement of the at least one positive electrode 152 and the at least one negative or ground electrode 154 about a central axis of the medical device 12. In some embodiments, the positive electrical pathway 142 and/or the negative or ground electrical pathway 144 may be substantially aligned with and/or parallel to the central axis of the medical device 12.

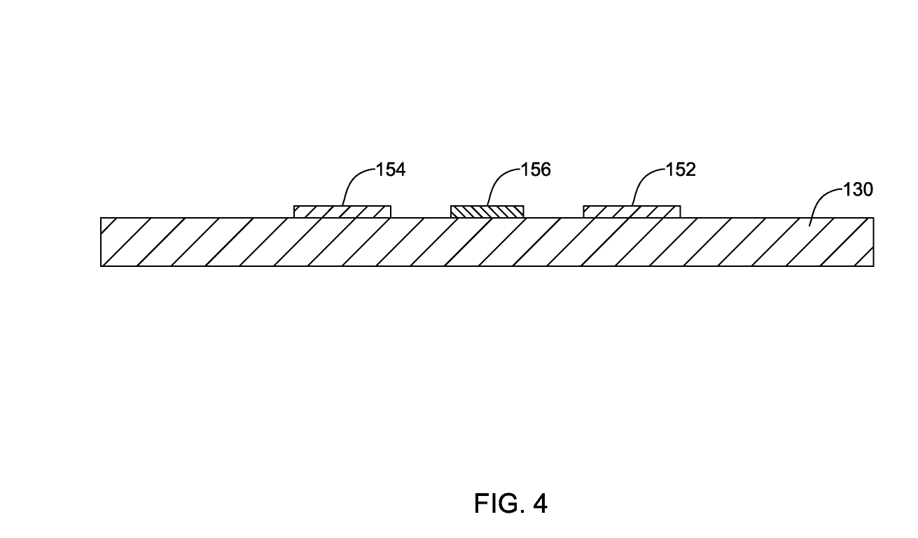

In some embodiments, a printed ablation electrode assembly 150 may be formed from a printable ink containing metallic nanoparticles. Forming a printed ablation electrode assembly 150 in this manner may provide a printed ablation electrode assembly 150 having increased flexibility and a very low profile on the outer surface of the expandable member 130 and/or the elongate tubular member or catheter shaft 122 due to the thinness of the printable ink, as seen in FIG. 4 for example. Delivery robustness may be improved and delivery forces may be reduced due to a lack of “catch points” created when a higher profile structure (i.e., electrode) is adhesively affixed to an expandable member. In some previous attempts to manufacture a printed ablation electrode assembly, these structures were difficult to navigate through the anatomy and/or could damage a balloon that was associated with the structure. The thin, flexible construction of a printed ablation electrode assembly 150 may provide improved performance.

In some embodiments, a temperature sensor 156 may be printed directly on the outer surface of the expandable member 130. In some embodiments, a temperature sensor 156 may be printed directly on the outer surface of the expandable member 130 between and/or aligned with the at least one positive electrode 152 and the at least one negative or ground electrode 154. In some embodiments, a temperature sensor 156 may be electrically coupled to the negative or ground electrical pathway 144. In some embodiments, a temperature sensor 156 may be electrically coupled to the negative or ground electrical pathway 144 through the at least one negative or ground electrode 154. In at least some embodiments, a temperature sensor 156 may be electrically coupled with a sensor electrical pathway 146 printed directly on the outer surface of the expandable member 130. In some embodiments, the sensor electrical pathway 146 may be electrically coupled to a corresponding electrical pathway on the elongate tubular member or catheter shaft 122. In some embodiments, the sensor electrical pathway 146 may be electrically coupled to a corresponding electrical pathway printed directly on the elongate tubular member or catheter shaft 122. In some embodiments, the sensor electrical pathway 146 (along with the corresponding electrical pathway on the elongate tubular member or catheter shaft 122) may be configured to supply positive electrical energy from the control unit 16 to the temperature sensor 156.

In some embodiments, a temperature sensor 156 may be formed from a printable ink containing silicon nanoparticles. In some embodiments, a temperature sensor 156 may be formed from a printable ink containing graphene or other suitable materials. Forming a temperature sensor 156 in this manner may provide a temperature sensor 156 having increased flexibility and a very low profile on the outer surface of the expandable member 130 due to the thinness of the printable ink, as seen in FIG. 4 for example. In some embodiments, forming a temperature sensor 156 in this manner may eliminate the presence of thick, inflexible ceramic structure(s) sometimes found in prior temperature sensors. In some previous constructions, a temperature sensor (e.g., thermistor) may have been the highest profile component on the expandable member. In some cases, these structures were difficult to navigate through the anatomy, corners rubbed and/or abraded a balloon upon which the structures were disposed, and/or made folding the balloon for insertion and withdrawal more difficult, resulting in compromised delivery forces and robustness upon delivery. The thin, flexible construction of a printed temperature sensor 156 may provide improved performance and robustness. In some embodiments, a temperature sensor 156 may be arranged in a sinusoidal or wave-like orientation on the outer surface of the expandable member 130, which may provide increased sensing performance while maintaining or improving flexibility on an expandable member 130. Other arrangements for a temperature sensor 156, such as longitudinally oriented, circumferentially oriented, helically oriented, etc. are also contemplated.

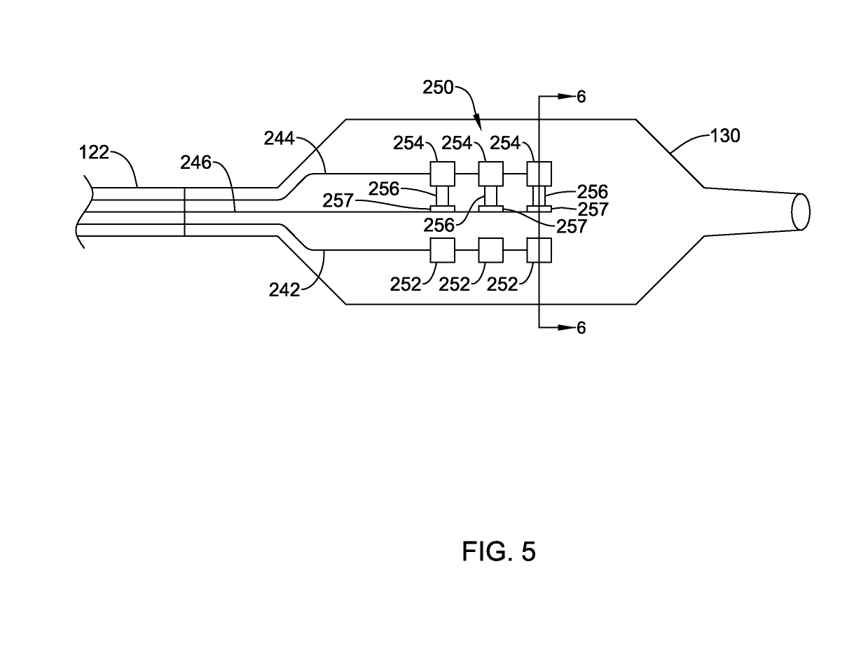

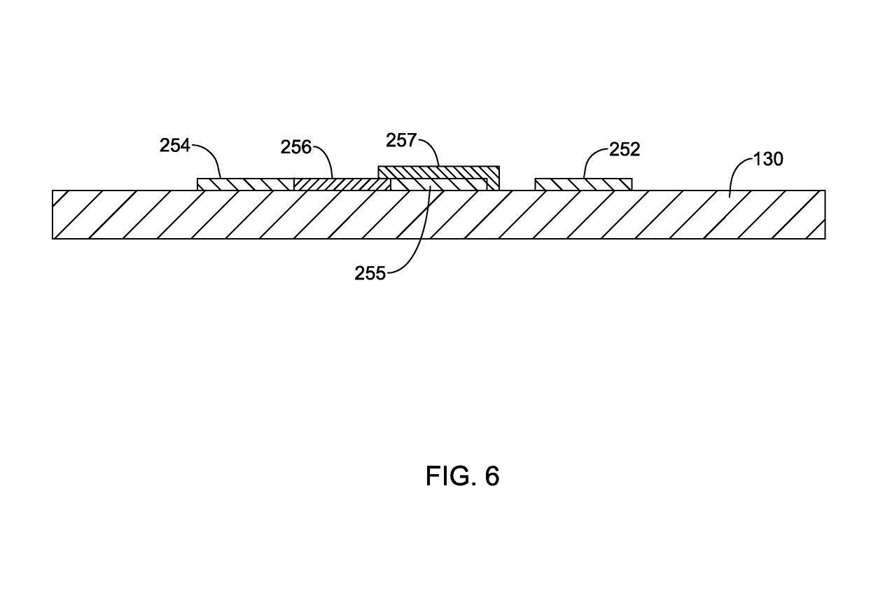

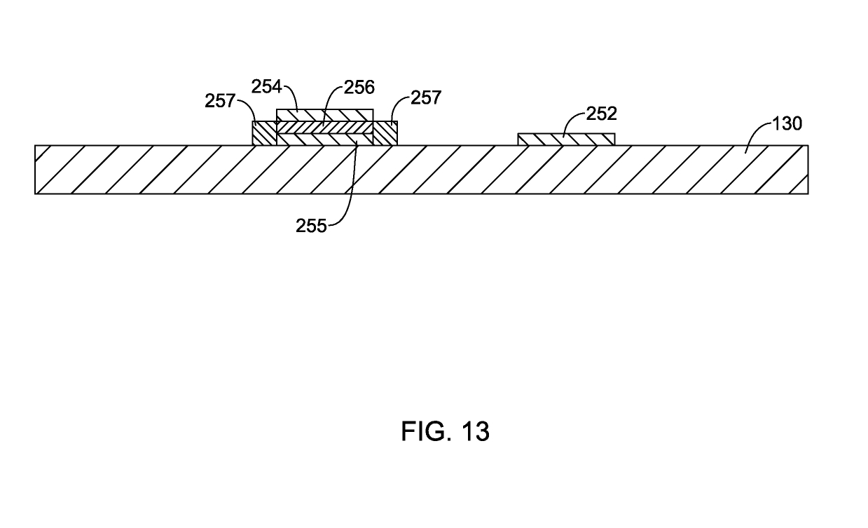

In some embodiments, as seen in FIGS. 5-6 for example, a printed ablation electrode assembly 250 may include a plurality of positive electrodes 252 printed directly on the outer surface of the expandable member 130 in electrical communication with the positive electrical pathway 242. In some embodiments, a printed ablation electrode assembly 250 may include a plurality of negative or ground electrodes 254 printed directly on the outer surface of the expandable member 130 in electrical communication with the negative or ground electrical pathway 244. In some embodiments, the plurality of positive electrodes 252 and the plurality of negative of ground electrodes 254 may be arranged in pairs. In some embodiments, a temperature sensor 256 may be printed directly on the outer surface of the expandable member 130. In some embodiments, a plurality of temperature sensors 256 may be printed directly on the outer surface of the expandable member 130, between and/or aligned with opposing pairs of positive and negative or ground electrodes as seen in FIG. 5, for example. In some embodiments, the plurality of temperature sensors 256 may be electrically coupled to a sensor electrical pathway 246. In some embodiments, the plurality of temperature sensors 256 may be electrically coupled to the sensor electrical pathway 246 through a sensor electrode 255, which may be electrically isolated and/or insulated from the plurality of positive electrodes 252 by a polymer isolating layer 257, as seen in FIG. 6. An alternative construction is shown illustratively in FIG. 13, wherein the sensor electrode 255 may be printed directly on the outer surface of the expandable member 130, the temperature sensor 256 may be printed directly onto the sensor electrode 255, and the negative or ground electrode 254 may be printed directly onto the temperature sensor 256 in a layered configuration. Other elements or details not expressly explained may be substantially similar to other disclosed embodiments or configurations discussed herein. The skilled person will recognize that other configurations are also possible.

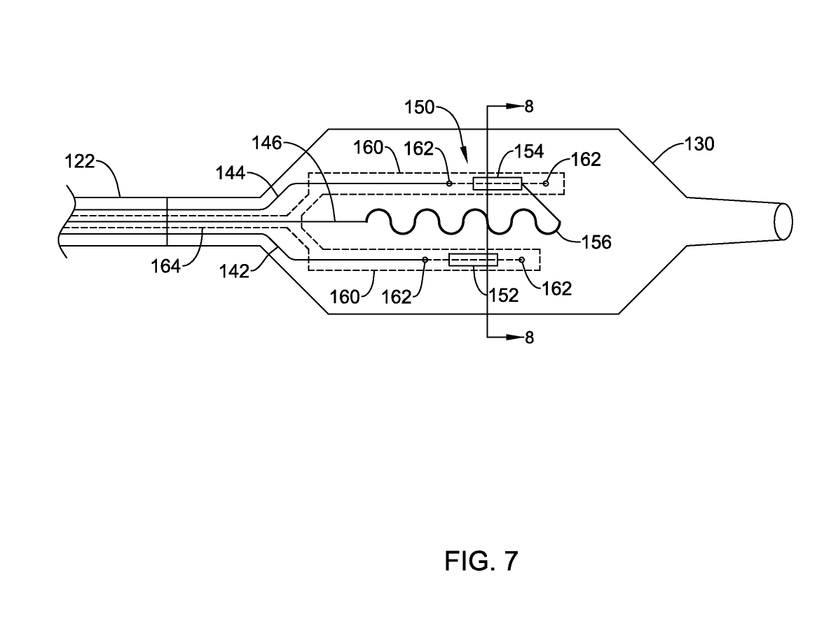

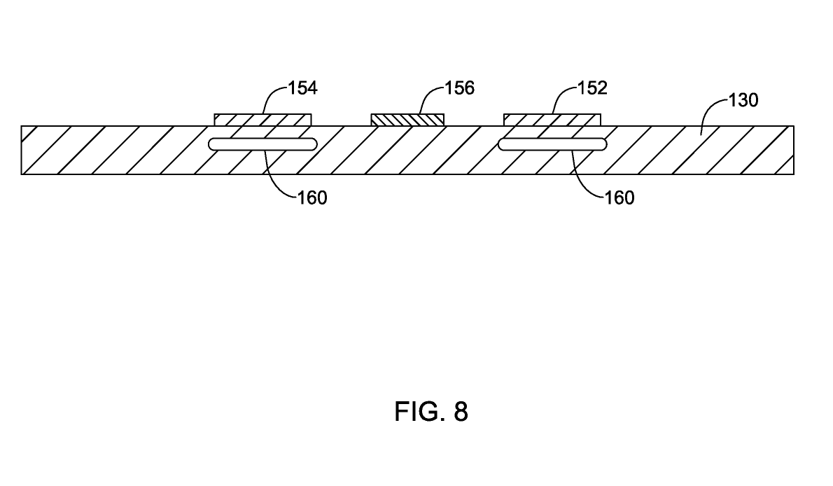

In some embodiments, the expandable member 130 may be an inflatable balloon. In at least some of these embodiments, the inflatable balloon may include at least one lumen 160 formed within a wall of the inflatable balloon under and longitudinally aligned with each of the at least one positive electrode 152 and/or the at least one negative or ground electrode 154, as seen in FIGS. 7-8 for example. In some embodiments where the at least one lumen 160 includes two or more total lumens 160, the two or more total lumens 160 may join or merge together to form a single supply lumen 164 at a proximal end of the inflatable balloon. In some embodiments, the single supply lumen 164 may be fluidly connected to a corresponding supply lumen within the elongate tubular member or catheter shaft 122, which may be fluidly connected to a source of fluid. In some embodiments, each of the at least one lumen 160 formed within a wall of the inflatable balloon may include at least one discharge aperture 162 through the outer surface of the inflatable balloon, as seen in FIG. 7 for example.

In some embodiments, a cooled biocompatible fluid may be delivered under positive pressure from the source of fluid through the supply lumen within the elongate tubular member or catheter shaft 122 to the single supply lumen 164. The cooled biocompatible fluid may be delivered under positive pressure from the single supply lumen 164 into the at least one lumen 160, where the cooled biocompatible fluid may pass under the at least one positive electrode 152 and/or the at least one negative or ground electrode 154 and be ejected through the at least one discharge aperture 162. The cooled biocompatible fluid may serve as a cooling fluid for the at least one positive electrode 152 and/or the at least one negative or ground electrode 154, and/or the tissue being treated by the printed ablation electrode assembly 150 to reduce and/or prevent damage to surface tissue(s) of a vessel wall (i.e., the endothelial layer) while permitting RF energy to ablate nervous tissue (i.e., renal nerves, etc.) beneath the surface tissue(s) of the vessel wall. In some embodiments, the at least one discharge aperture 162 may be disposed distal of the at least one positive electrode 152 and/or the at least one negative or ground electrode 154. In some embodiments, the at least one discharge aperture 162 may be disposed proximal of the at least one positive electrode 152 and/or the at least one negative or ground electrode 154. In some embodiments, the at least one discharge aperture 162 may be disposed both proximal and distal of the at least one positive electrode 152 and/or the at least one negative or ground electrode 154. In other words, in some embodiments, each lumen 160 formed within a wall of the inflatable balloon may include a plurality of discharge apertures 162. Additionally, in some embodiments, each lumen 160 associated with an electrode may be formed as a pair (or more) of lumens associated with the electrode, each having one or more discharge apertures.

In some embodiments, the cooled biocompatible fluid may include saline solution, anti-inflammatory drugs or other pharmaceuticals, and the like. In some embodiments, the biocompatible fluid may include a heat-shock protein, such as Hsp104 and/or Hsp70. The heat-shock protein Hsp104 and/or Hsp70 may improve the survival of cells exposed to high temperatures and other stresses. Hsp104 and/or Hsp70 may help to repair and/or restore the splicing of intervening sequences from mRNA precursers that has been damaged by heat shock.

In some embodiments, the at least one lumen 160 formed within a wall of the inflatable balloon may be aligned generally parallel with the outer surface of the inflatable balloon, although this is not required. In some embodiments, the at least one lumen 160 formed within a wall of the inflatable balloon may be aligned generally parallel with a longitudinal axis of the elongate tubular member or catheter shaft 122 and/or the inflatable balloon. In some embodiments, the at least one lumen 160 may have a generally ovoid or oblong cross-sectional shape, as seen in FIG. 8. However, other suitable cross-sectional shapes and configurations, such as, but not limited to, round, square, rectangular, triangular, polygonal, irregular, complex, or other suitable shapes, are also contemplated.

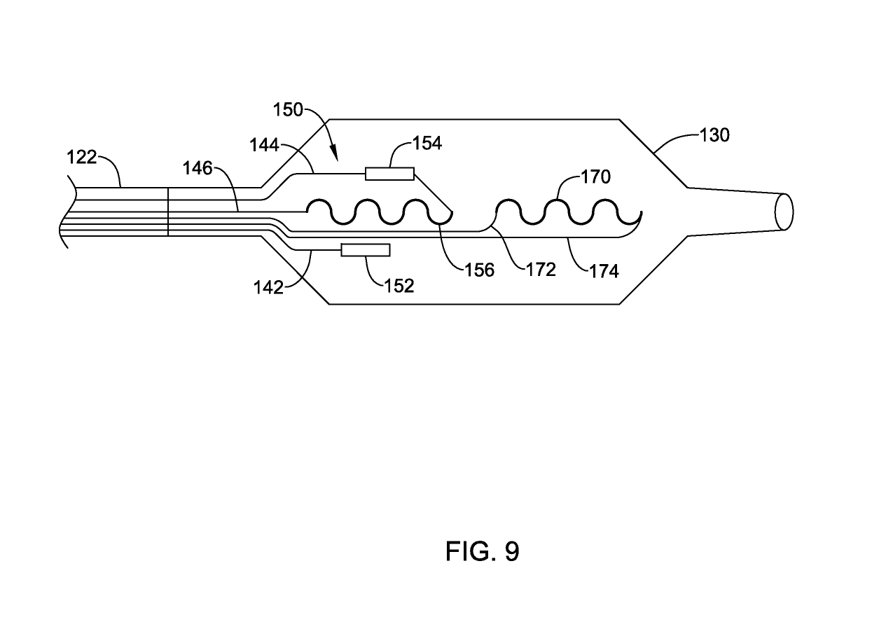

In some embodiments, a medical device 12 may include a second temperature sensor 170 printed directly on the outer surface of the expandable member 130 and spaced distally from the printed ablation electrode assembly 150, as seen for example in FIG. 9. In general, the second temperature sensor 170 may be substantially similar to the temperature sensor 156 described above, although variations in construction, shape, materials, etc. are contemplated. In some embodiments, a second temperature sensor 170 may be electrically coupled to a second negative or ground electrical pathway 174 printed directly on the outer surface of the expandable member 130. In some embodiments, the second negative or ground electrical pathway 174 may be electrically coupled to a corresponding electrical pathway on the elongate tubular member or catheter shaft 122. In some embodiments, a second temperature sensor 170 may be electrically coupled with a second sensor electrical pathway 172 printed directly on the outer surface of the expandable member 130. In some embodiments, the second sensor electrical pathway 172 may be electrically coupled to a corresponding electrical pathway on the elongate tubular member or catheter shaft 122.

In some embodiments, the second sensor electrical pathway 172 (along with the corresponding electrical pathway on the elongate tubular member or catheter shaft 122) may be configured to supply positive electrical energy from the control unit 16 to the second temperature sensor 170. In some embodiments, the second sensor electrical pathway 172 and/or the second negative or ground electrical pathway 174 may pass between and/or adjacent to the at least one positive electrode 152 and the at least one negative or ground electrode 154 on the outer surface of the expandable member 130. In some embodiments, the second sensor electrical pathway 172 and/or the second negative or ground electrical pathway 174 may be covered or electrically insulated to prevent interference from the at least one positive electrode 152 and/or the at least one negative or ground electrode 154. In some embodiments, the second temperature sensor 170 may be configured to detect heat transferred distally by fluid flow within a vessel lumen (i.e., at a treatment site) if the expandable member 130 and/or the at least one positive electrode 152 and the at least one negative or ground electrode 154 is not in close contact with a wall of the vessel lumen. An expandable member 130 and/or a printed ablation electrode assembly 150 that is/are in direct contact with a vessel wall may transfer most of its heat to adjacent tissue, with very little heat escaping or being carried downstream. The second temperature sensor 170 spaced distally from the printed ablation electrode assembly 150 may be used to determine if good and/or direct contact between the printed ablation electrode assembly 150 and the vessel wall is being achieved. If the second temperature sensor 170 determines that good and/or direct contact between the printed ablation electrode assembly 150 and the vessel wall has not been achieved, the expandable member 130 may be expanded further by an appropriate means of doing so to achieve improved vessel apposition. Similarly, a second temperature sensor 170 may permit a compliant inflatable balloon to be inflated to a lower pressure while maintaining good and/or proper contact between the printed ablation electrode assembly 150 and the vessel wall.

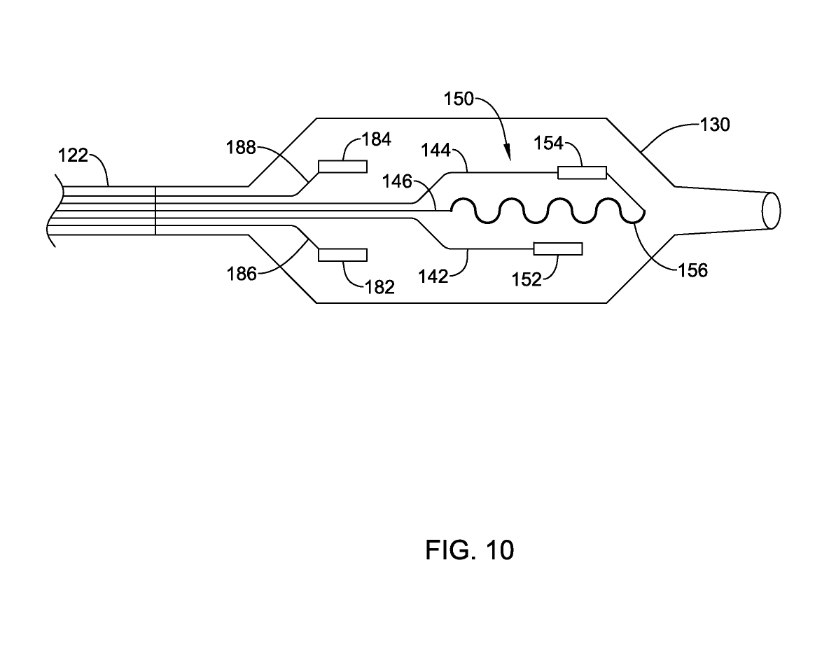

In some embodiments, a medical device 12 may include a second positive electrode 182 printed directly on the outer surface of the expandable member 130 and distinct from the printed ablation electrode assembly 150 and/or the at least one positive electrode 152. In some embodiments, a medical device 12 may include a second negative or ground electrode 184 printed directly on the outer surface of the expandable member 130 and distinct from the printed ablation electrode assembly 150 and/or the at least one negative or ground electrode 154. In some embodiments, the second positive electrode 182 and/or the second negative or ground electrode 184 may be disposed proximally of, and/or spaced proximally from, the printed ablation electrode assembly 150, the at least one positive electrode 152, and/or the at least one negative or ground electrode 154, as seen for example in FIG. 10.

In general, the second positive electrode 182 and/or the second negative or ground electrode 184 may be substantially similar to the at least one positive electrode 152, and/or the at least one negative or ground electrode 154 described above, although variations in construction, shape, materials, etc. are contemplated. In some embodiments, a second negative or ground electrode 184 may be electrically coupled to a second electrode negative or ground electrical pathway 188 printed directly on the outer surface of the expandable member 130. In some embodiments, the second electrode negative or ground electrical pathway 188 may be electrically coupled to a corresponding electrical pathway on the elongate tubular member or catheter shaft 122. In some embodiments, a second positive electrode 182 distinct from the at least one positive electrode may be electrically coupled with a second positive electrical pathway 186 printed directly on the outer surface of the expandable member 130. In some embodiments, the second positive electrical pathway 186 may be electrically coupled to a corresponding electrical pathway on the elongate tubular member or catheter shaft 122.

In some embodiments, the second positive electrical pathway 186 (along with the corresponding electrical pathway on the elongate tubular member or catheter shaft 122) may be configured to supply positive electrical energy from the control unit 16 to the second positive electrode 182. In some embodiments, the positive electrical pathway 142, the negative or ground electrical pathway 144, and/or the sensor electrical pathway 146 may pass between and/or adjacent to the second positive electrode 182 and the second negative or ground electrode 184 on the outer surface of the expandable member 130. In some embodiments, the positive electrical pathway 142, the negative or ground electrical pathway 144, and/or the sensor electrical pathway 146 may be covered or electrically insulated to prevent interference from the second positive electrode 182 and/or the second negative or ground electrode 184.

In some embodiments, the second positive electrode 182 and the second negative or ground electrode 184 may form a pair of electrodes. In some embodiments, the second positive electrode 182 and the second negative or ground electrode 184 may be configured to operate at a reduced power or intensity compared to the printed ablation electrode assembly 150 and/or the at least one positive electrode 152 and the at least one negative or ground electrode 154. In some embodiments, the second positive electrode 182 and the second negative or ground electrode 184 may be configured to disrupt nervous activity at a location proximal, or a different position radially, of a treatment site being treated by the printed ablation electrode assembly 150. In some embodiments, disruption of nervous activity proximal, or a different position radially, of the treatment site being treated by the relatively higher power printed ablation electrode assembly 150 may reduce or prevent transmission of pain signals to a patient's brain during ablation of nervous tissue at the treatment site being treated by the printed ablation electrode assembly 150.

In addition or alternatively, a medical device 12 having a printed ablation electrode assembly 150 and a second positive electrode 182 and a second negative or ground electrode 184 printed directly on an outer surface of an inflatable balloon may further include at least one lumen 160 formed within a wall of the inflatable balloon under and longitudinally aligned with each of the at least one positive electrode 152, the at least one ground electrode 154, the second positive electrode 182, and the second negative or ground electrode 184, as described above with respect to FIG. 7. As such, some or all of the elements and features disclosed and described with respect to FIGS. 7 and 10 are expressly considered as being usable in combination with each other.

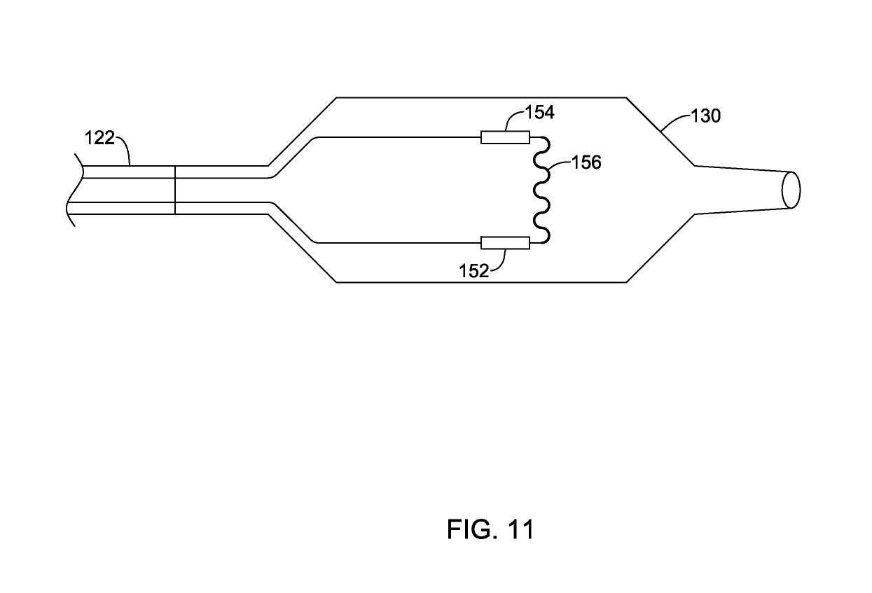

In some embodiments, a temperature sensor 156 may be formed as a negative temperature coefficient (NTC) thermistor printed directly on the outer surface of the expandable member 130. In some embodiments, a temperature sensor 156 may be formed as a self-regulating positive temperature coefficient (PTC) electrode printed directly on the outer surface of the expandable member 130 connecting a positive electrical pathway 142 and a negative or ground electrical pathway 144, as seen for example in FIG. 11. In some embodiments, a positive temperature coefficient (PTC) electrode may form a resistive heating element. In some embodiments, a positive temperature coefficient (PTC) electrode may form a thermistor. In some embodiments, a positive temperature coefficient (PTC) electrode may form a combined heating element and thermistor, and thus be capable of self-regulation.

Generally, both negative temperature coefficient (NTC) and positive temperature coefficient (PTC) electrodes or thermistors work due to a change in electrical resistance as a function of temperature. In some cases, NTC thermistors may be made from a semiconductor material such as a sintered metal oxide. Raising the temperature of a semiconductor increases the number of active charge carriers into the conduction band. The more charge carriers that are available, the more current a material can conduct. The conductor can be both an n-type or p-type conductor. In effect, the electrical resistance of an NTC thermistor is lowered at increasing temperatures.

Most PTC thermistors are of the “switching” type, which means that their electrical resistance rises suddenly at a certain critical temperature. In some cases, PTC thermistors may be made from a doped polycrystalline ceramic containing barium titanate (BaTiO3) and/or other compounds. The dielectric constant of this ferroelectric material varies with temperature. Below the Curie point temperature, the high dielectric constant prevents the formation of potential barriers between the crystal grains, leading to a low electrical resistance. In this region the device may have a small negative temperature coefficient. At the Curie point temperature, the dielectric constant drops sufficiently to allow the formation of potential barriers at the grain boundaries, and the electrical resistance increases sharply. Since current flowing through a resistor generates heat, a “switching” PTC thermistor may be used as a self-regulating combined heating element and thermistor by choosing a switching point at a preferred operating temperature of the device.

Another type of thermistor is a silistor, or a thermally sensitive silicon resistor. Silistors employ silicon as the semiconductive component material. In contrast to the “switching” type PTC thermistors, silistors have an almost linear resistance-temperature characteristic. Both silistors and “switching” type PTC thermistors raise the electrical resistance at increasing temperatures.

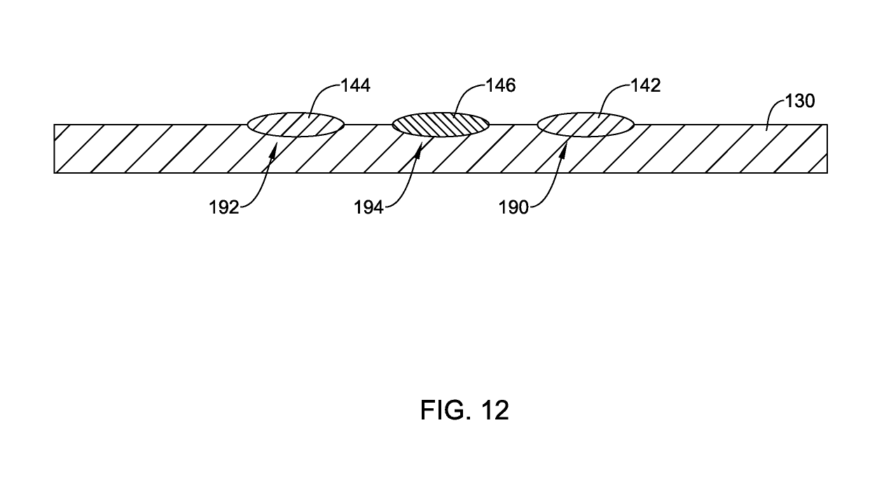

In some embodiments, printing electrical pathways, electrodes, temperature sensors, and the like, as described herein, may be made easier by minor modifications to the substrate upon which these elements are printed. Printable inks, being liquid suspensions and flowable by nature, may undesirably run or spread out on some substrates. To reduce the tendency of the ink(s) to move away from their intended location of deposition, barriers may be used to “direct” the ink(s) into a desired location. In some embodiments, shallow recessed channels may be formed or cut into an outer surface of an expandable member 130, as shown illustratively in FIG. 12, and/or the elongate tubular member or catheter shaft 122. In some embodiments, the shallow recessed channels may be cut into the outer surface of the expandable member 130 and/or the elongate tubular member or catheter shaft 122 using laser ablation, machine tools, chemical dissolution, or other suitable means. In some embodiments, the recessed channels may be formed during manufacturing of the expandable member 130 (i.e., during a balloon molding process, for example). Printable ink(s) may be deposited directly onto the outer surface of the expandable member 130 and/or the elongate tubular member or catheter shaft 122 within the recessed channels. In some embodiments, the recessed channels may serve or help to protect the printed surface, thereby providing improved robustness thereof. In some embodiments, surface tension of the exposed portion of the printable ink(s) may cooperate with the recessed channels to hold the ink(s) in place. Alternatively, a fine laser beam could be used to change the surface tension along a line without making a recessed channel. The printable ink(s) may then be self-confined along this line. In addition or alternatively, polymer shoulders may be added to the outer surface of the expandable member 130 and/or the elongate tubular member or catheter shaft 122 to form channels on and/or above the outer surface of the expandable member 130 and/or the elongate tubular member or catheter shaft 122. In some embodiments, the polymer shoulders may be integrally formed with a wall of the expandable member 130 and/or the elongate tubular member or catheter shaft 122. In some embodiments, the polymer shoulders may be printed, deposited, or otherwise added onto the outer surface of the expandable member 130 and/or the elongate tubular member or catheter shaft 122.

In some embodiments, a positive electrical pathway 142 may be printed directly on the outer surface of an expandable member 130, which may be an inflatable balloon, in a first recessed channel 190 formed in the outer surface of the inflatable balloon or expandable member 130. In some embodiments, the positive electrical pathway 142 may be partially recessed below the outer surface of the expandable member 130. In other words, the positive electrical pathway 142 may extend a fraction of its thickness above the outer surface of the expandable member 130. In some embodiments, the positive electrical pathway 142 may be substantially flush with the outer surface of the expandable member 130. In some embodiments, the positive electrical pathway 142 may be recessed below the outer surface of the expandable member 130. In some embodiments, the positive electrical pathway 142 may be substantially covered or electrically insulated along its length.

In some embodiments, a negative or ground electrical pathway 144 may be printed directly on the outer surface of an expandable member 130, which may be an inflatable balloon, in a second recessed channel 192 formed in the outer surface of the inflatable balloon or expandable member 130. In some embodiments, the negative or ground electrical pathway 144 may be partially recessed below the outer surface of the expandable member 130. In other words, the negative or ground electrical pathway 144 may extend a fraction of its thickness above the outer surface of the expandable member 130. In some embodiments, the negative or ground electrical pathway 144 may be substantially flush with the outer surface of the expandable member 130. In some embodiments, the negative or ground electrical pathway 144 may be recessed below the outer surface of the expandable member 130. In some embodiments, the negative or ground electrical pathway 144 may be substantially covered or electrically insulated along its length.

In some embodiments, a sensor electrical pathway 146 may be printed directly on the outer surface of an expandable member 130, which may be an inflatable balloon, in a third recessed channel 194 formed in the outer surface of the inflatable balloon or expandable member 130. In some embodiments, the sensor electrical pathway 146 may be partially recessed below the outer surface of the expandable member 130. In other words, the sensor electrical pathway 146 may extend a fraction of its thickness above the outer surface of the expandable member 130. In some embodiments, the sensor electrical pathway 146 may be substantially flush with the outer surface of the expandable member 130. In some embodiments, the sensor electrical pathway 146 may be recessed below the outer surface of the expandable member 130. In some embodiments, the sensor electrical pathway 146 may be substantially covered or electrically insulated along its length.

In some embodiments, at least one positive electrode 152 may be printed directly on the outer surface of an expandable member 130, which may be an inflatable balloon, in a recessed channel (first recessed channel 190, or a separate, fourth recessed channel) formed in the outer surface of the inflatable balloon or expandable member 130. In some embodiments, the at least one positive electrode 152 may be partially recessed below the outer surface of the expandable member 130. In other words, the at least one positive electrode 152 may extend a fraction (less than a whole) of its thickness above the outer surface of the expandable member 130. In some embodiments, the at least one positive electrode 152 may be substantially flush with the outer surface of the expandable member 130. In some embodiments, the at least one positive electrode 152 may be recessed below the outer surface of the expandable member 130. In some embodiments, the at least one positive electrode 152 may be substantially covered or electrically insulated along a portion of its length.

In some embodiments, at least one negative or ground electrode 154 may be printed directly on the outer surface of an expandable member 130, which may be an inflatable balloon, in a recessed channel (second recessed channel 192, or a separate, fifth recessed channel) formed in the outer surface of the inflatable balloon or expandable member 130. In some embodiments, the at least one negative or ground electrode 154 may be partially recessed below the outer surface of the expandable member 130. In other words, the at least one negative or ground electrode 154 may extend a fraction of its thickness (less than a whole) above the outer surface of the expandable member 130. In some embodiments, the at least one negative or ground electrode 154 may be substantially flush with the outer surface of the expandable member 130. In some embodiments, the at least one negative or ground electrode 154 may be recessed below the outer surface of the expandable member 130. In some embodiments, the at least one negative or ground electrode 154 may be substantially covered or electrically insulated along a portion of its length.

In some embodiments, a temperature sensor 156 may be printed directly on the outer surface of an expandable member 130, which may be an inflatable balloon, in a recessed sensor channel formed in the outer surface of the inflatable balloon or expandable member 130 between the at least one positive electrode 152 and the at least one negative or ground electrode 154. In some embodiments, the recessed sensor channel may extend from the first recessed channel 190 to the second recessed channel 192 or from the fourth recessed channel to the fifth recessed channel. In some embodiments, the temperature sensor 156 may be partially recessed below the outer surface of the expandable member 130. In other words, the temperature sensor 156 may extend a fraction of its thickness (less than a whole) above the outer surface of the expandable member 130. In some embodiments, the temperature sensor 156 may be substantially flush with the outer surface of the expandable member 130. In some embodiments, the temperature sensor 156 may be recessed below the outer surface of the expandable member 130. In some embodiments, the temperature sensor 156 may be substantially covered or electrically insulated along a portion of its length.

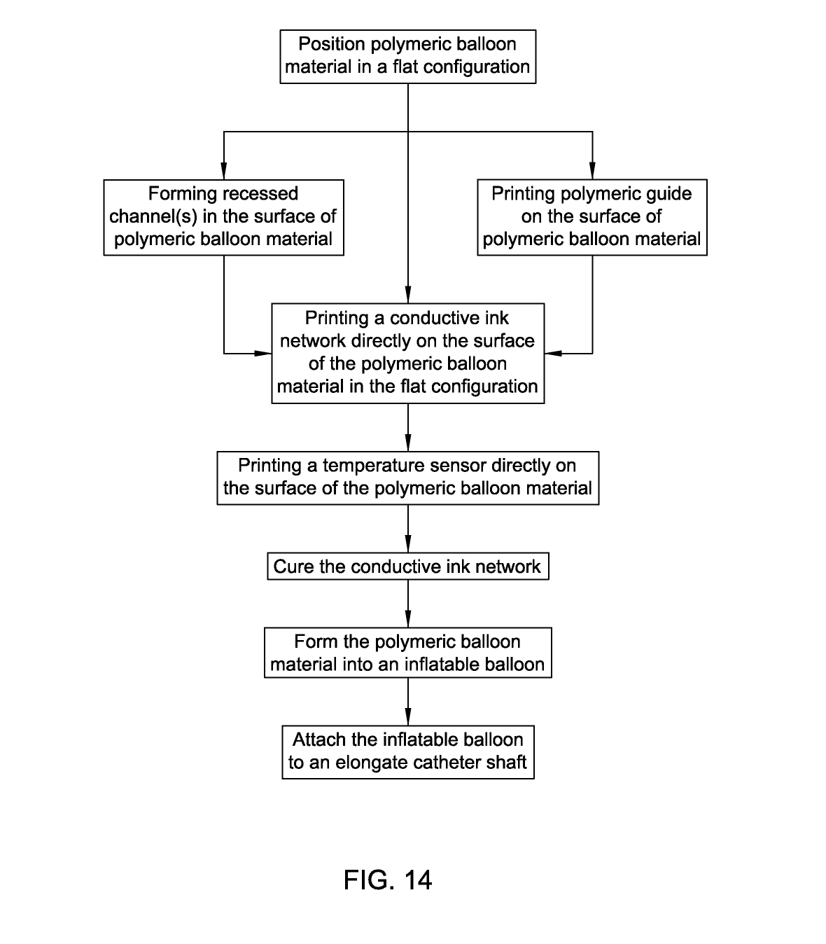

In some embodiments, a method of manufacturing a medical device 12 for sympathetic nerve ablation, shown illustratively in FIG. 14, may include printing a conductive ink network directly on a surface of a polymeric balloon material and/or an elongate tubular member or catheter shaft. In some embodiments, a conductive ink network may be formed from a nanoparticle suspension, wherein metallic particles are encapsulated in an organic binder. In some embodiments, a method of manufacturing a medical device 12 for sympathetic nerve ablation may include printing at least one temperature sensor directly on the surface of the polymeric balloon material. In some embodiments, a method of manufacturing a medical device 12 for sympathetic nerve ablation may include positioning a polymeric balloon material in a flat configuration prior to printing a conductive ink network directly on the surface of the polymeric balloon material. In some embodiments, a method of manufacturing a medical device 12 for sympathetic nerve ablation may include printing a conductive ink network directly on the surface of the polymeric balloon material in the flat configuration. In some embodiments, a method of manufacturing a medical device 12 for sympathetic nerve ablation may include forming the polymeric balloon material into an inflatable balloon. In some embodiments, before forming the polymeric balloon material in an inflatable balloon, a method of manufacturing a medical device 12 for sympathetic nerve ablation may include curing the conductive ink network. In some embodiments, curing the conductive ink network may include heating the conductive ink network to decompose the organic binder. In some embodiments, after forming the polymeric balloon material into an inflatable balloon, a method of manufacturing a medical device 12 for sympathetic nerve ablation may include attaching the inflatable balloon to an elongate tubular member or catheter shaft.

In some embodiments, a method of manufacturing a medical device 12 for sympathetic nerve ablation may include forming at least one recessed channel in the surface of the polymeric balloon material and/or the elongate tubular member or catheter shaft, before printing the conductive ink network, wherein the conductive ink network is then printed within the at least one recessed channel. In some embodiments, a method of manufacturing a medical device 12 for sympathetic nerve ablation may include printing a polymer guide on the surface of the polymeric balloon material and/or the elongate tubular member or catheter shaft, before printing the conductive ink network, wherein the conductive ink network is then printed within the polymer guide.

In use, a medical device 12 may be advanced intravascularly to one or more treatment sites within a vessel lumen over and/or along a guidewire 102, within a guide sheath or catheter 14, or both. Components of the medical device 12 formed using printed conductive ink may improve overall device profile, flexibility, navigability, and/or robustness of the medical device 12. An expandable member 130, which may be an inflatable balloon, is expanded from a collapsed configuration to a first expanded configuration at a first treatment site, which may be within a renal artery for example, to press electrodes positioned on the outer surface of the expandable member 130 into contact with a wall of the vessel lumen at the first treatment site. In the first expanded configuration, the expandable member 130 may have a first maximum outer diameter or extent. In some embodiments, for example in the case of an inflatable balloon, the expandable member 130 may be expanded by pressurizing fluid from about 1-10 atm. A first treatment procedure may then begin. Electrical energy may be supplied from a control unit 16 to the medical device 12. Ablation energy, for example RF energy, may be transmitted from at least one positive electrode 152, through the target tissue, to at least one negative or ground electrode 154, the electrodes being printed directly on an outer surface of the expandable member 130, to specifically cause ablation of target nervous tissue disposed radially outward from the vessel lumen while minimizing tissue damage to the endothelial layer (i.e., the inner surface) of the vessel. A temperature sensor printed directly on the outer surface of the expandable member 130 may be useful to detect conditions such as tissue overheating, improper contact between the electrodes and the vessel wall, and/or improper cooling, if the device is so equipped.

Following a first treatment procedure, the expandable member 130 may be collapsed back to a collapsed configuration, and the medical device 12 may be refracted within the guide sheath or catheter 14. Additionally or alternatively, the medical device 12 may be repositioned to a second treatment site, where the expandable member 130 may be expanded again from the collapsed configuration to a second expanded configuration, to press the electrodes positioned on the outer surface of the expandable member 130 into contact with a wall of the vessel lumen at the second treatment site. In some embodiments, for example in the case of an inflatable balloon, the expandable member 130 may be expanded by pressurizing fluid from about 1-10 atm. In the second expanded configuration, the expandable member 130 may have a second maximum outer diameter or extent. In some embodiments, the second maximum outer diameter or extent may be different from the first maximum outer diameter or extent. For example, in some embodiments, the second maximum outer diameter or extent may be greater than the first maximum outer diameter or extent. In some embodiments, the second maximum outer diameter or extent may be less than the first maximum outer diameter or extent. A second treatment procedure may then commence at the second treatment site, utilizing the same steps, actions, and structures as the first treatment procedure. Additional treatment procedures at additional treatment sites are also contemplated.

A lack of high profile protrusion(s) and improved flexibility from the use of a printed ablation electrode assembly 150 may make refraction of the medical device 12 into the guide sheath or catheter 14 easier and/or permit the use of a smaller diameter guide sheath or catheter 14 than would otherwise be required. Additionally, the lack of high profile protrusion(s) and improved flexibility may positively affect the foldability characteristics of the expandable member 130, both for improved delivery and for improved withdrawal, with lower delivery forces required.

The materials that can be used for the various components of the medical device 12 (and/or other devices disclosed herein) may include those commonly associated with medical devices. For simplicity purposes, the following discussion makes reference to the medical device 12. However, this is not intended to limit the devices and methods described herein, as the discussion may be applied to elements of the medical device 12 and/or other similar tubular members and/or expandable members and/or components of tubular members and/or expandable members disclosed herein.

The medical device 12 and the various components thereof may be made from a metal, metal alloy, polymer (some examples of which are disclosed below), a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear elastic and/or non-super-elastic nitinol does not display a substantial “superelastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear that the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also can be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (° C.) to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Some examples of nickel titanium alloys are disclosed in U.S. Pat. Nos. 5,238,004 and 6,508,803, which are incorporated herein by reference. Other suitable materials may include ULTANIUM™ (available from Neo-Metrics) and GUM METAL™ (available from Toyota). In some other embodiments, a superelastic alloy, for example a superelastic nitinol can be used to achieve desired properties.

In at least some embodiments, portions of the medical device 12 may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the medical device 12 in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the medical device 12 to achieve the same result.

In some embodiments, a degree of Magnetic Resonance Imaging (MRI) compatibility may be imparted into the medical device 12. For example, portions of device, may be made of a material that does not substantially distort the image and create substantial artifacts (i.e., gaps in the image). Certain ferromagnetic materials, for example, may not be suitable because they may create artifacts in an MRI image. In some of these and in other embodiments, portions of the medical device 12 may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A medical device for sympathetic nerve ablation at one or more treatment sites, comprising:
an elongate catheter shaft having a guidewire lumen extending therethrough and an expandable member disposed adjacent a distal end;
a printed ablation electrode assembly disposed on an outer surface of the expandable member, the printed ablation electrode assembly including a positive electrical pathway printed directly on the outer surface of the expandable member and a ground electrical pathway printed directly on the outer surface of the expandable member; and
a temperature sensor printed directly on the outer surface of the expandable member.

2. The medical device of claim 1, wherein the printed ablation electrode assembly includes at least one positive electrode printed directly on the outer surface of the expandable member in electrical communication with the positive electrical pathway, and at least one ground electrode printed directly on the outer surface of the expandable member in electrical communication with the ground electrical pathway.

3. The medical device of claim 2, wherein the temperature sensor is printed directly on the outer surface of the expandable member between the at least one positive electrode and the at least one ground electrode.

4. The medical device of claim 2, further including:
a positive electrical pathway printed directly on an outer surface of the elongate catheter shaft and in electrical communication with the positive electrical pathway printed directly on the outer surface of the expandable member; and
a ground electrical pathway printed directly on the outer surface of the elongate catheter shaft and in electrical communication with the ground electrical pathway printed directly on the outer surface of the expandable member.

5. The medical device of claim 2, further including a second temperature sensor printed directly on the outer surface of the expandable member and spaced distally from the printed ablation electrode assembly.

6. The medical device of claim 2, further including a second positive electrode distinct from the at least one positive electrode, and a second ground electrode distinct from the at least one ground electrode;
wherein the second positive electrode distinct from the at least one positive electrode and the second ground electrode distinct from the at least one ground electrode are disposed proximally of the at least one positive electrode and the at least one ground electrode, and are configured to operate at a reduced power compared to the at least one positive electrode and the at least one ground electrode.

7. The medical device of claim 2, wherein the expandable member is an inflatable balloon.

8. The medical device of claim 7, further including at least one lumen formed within a wall of the inflatable balloon under and longitudinally aligned with each of the at least one positive electrode and the at least one ground electrode.

9. The medical device of claim 8, wherein each of the at least one lumen formed within the wall of the inflatable balloon includes at least one discharge aperture through the outer surface of the inflatable balloon.

10. The medical device of claim 1, wherein the temperature sensor is a self-regulating positive temperature coefficient (PTC) electrode printed directed on the outer surface of the expandable member connecting the positive electrical pathway and the ground electrical pathway.

11. A medical device for sympathetic nerve ablation at one or more treatment sites, comprising:
an elongate catheter shaft having a guidewire lumen extending therethrough and an inflatable balloon disposed adjacent a distal end;
a positive electrical pathway printed directly on an outer surface of the inflatable balloon in a first recessed channel formed in the outer surface of the inflatable balloon;
a ground electrical pathway printed directly on the outer surface of the inflatable balloon in a second recessed channel formed in the outer surface of the inflatable balloon; and
a temperature sensor printed directly on the outer surface of the inflatable balloon.

12. The medical device of claim 11, wherein the temperature sensor is printed directly on the outer surface of the inflatable balloon in a third recessed channel formed in the outer surface of the inflatable balloon.

13. The medical device of claim 11, wherein the temperature sensor is a self-regulating positive temperature coefficient (PTC) electrode printed directed on the outer surface of the inflatable balloon connecting the positive electrical pathway and the ground electrical pathway.

14. The medical device of claim 11, including at least one positive electrode printed directly on the outer surface of the inflatable balloon in electrical communication with the positive electrical pathway, and at least one ground electrode printed directly on the outer surface of the inflatable balloon in electrical communication with the ground electrical pathway.

15. The medical device of claim 14, wherein the temperature sensor is printed directly on the outer surface of the inflatable balloon between the at least one positive electrode and the at least one ground electrode.

16. A method of manufacturing a medical device for sympathetic nerve ablation, comprising:
positioning a polymeric balloon material in a flat configuration;
printing a conductive ink network directly on a surface of the polymeric balloon material in the flat configuration;
printing at least one temperature sensor directly on the surface of the polymeric balloon material;
forming the polymeric balloon material into an inflatable balloon; and
attaching the inflatable balloon to an elongate catheter shaft.

17. The method of claim 16, further comprising:
before printing the conductive ink network, forming at least one recessed channel in the surface of the polymeric balloon material;
wherein the conductive ink network is printed within the at least one recessed channel.

18. The method of claim 16, further comprising:
before printing the conductive ink network, printing a polymer guide on the surface of the polymeric balloon material;
wherein the conductive ink network is printed within the polymer guide.

19. The method of claim 16, further comprising:
before forming the polymeric balloon material into the inflatable balloon, curing the conductive ink network.

20. The method of claim 16, wherein the conductive ink network is formed from a nanoparticle suspension having metallic nanoparticles encapsulated by an organic binder.
